(19) United States
(12) Patent Application Publication
(10) Pub. No.: US 2010/0286280 A1
(43) Pub. Date: Nov. 11, 2010

(54) INHIBITORS OF EPPIN/SEMENOGELIN BINDING AS MALE CONTRACEPTIVES

(75) Inventors: Michael G. O'Rand, Chapel Hill, NC (US); Esther Elaine Widgren, Chapel Hill, NC (US); Richard Richardson, Durham, NC (US); Brenda Temple, Chapel Hill, NC (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: The University of North Carolina at Chapel Hill, Chapel Hill, NC (US)

(21) Appl. No.: 12/680,339

(22) PCT Filed: Sep. 23, 2008
(86) PCT No.: PCT/US08/77312

§ 371 Date: Jul. 16, 2010

Related U.S. Application Data
(60) Provisional application No. 60/995,293, filed on Sep. 26, 2007.

(51) Int. Cl. A61K 31/16; A61P 15/16

(52) U.S. Cl. 514/616

(57) ABSTRACT

Compounds suitable for use in providing male contraception, an assay method for identifying such compounds, and methods of providing contraception using the compounds, are provided. The compounds described herein inhibit Eppin-semenogelin binding, and inhibit forward motility of sperm in humans and other primates. The assays identify compounds which both inhibit eppin-semenogelin binding and inhibit sperm motility, and can be carried out in a high throughput manner, using labeled recombinant Eppin and semenogelin. The compounds can be used in oral or transdermal compositions to temporarily and reversibly inhibit male fertility. They can also be used in addition to, or in place of, spermicides in spermicidal compositions, such as those used in conjunction with condoms, diaphragms, and spermicidal jellies.

### BACKGROUND OF THE INVENTION

Over the last 50 years, contraception has had a major impact on human society and influenced the world wide distribution of family sizes and the variability of fertility rates (Bongaarts and Watkins, 1996; Bongaarts, 1997). This impact can be largely attributed to female contraceptive methods, their availability and economic and social costs.

Male contraception, on the other hand, has had much less of a global impact, being largely limited to condoms and vasectomy (Nass and Strauss, 2004). Female hormonal contraceptives work through the mechanism of anovulation and the goal of male hormonal contraceptive research is analogous, namely the suppression of spermatogenesis to produce azoospermia. However, achievement of this goal in a reliable way for a diverse population of men is still many years away (Grimes et al., 2005; Potts, 1996).

Even further away is the dream of a non-hormonal male contraceptive in which it may be envisioned that spermatozoa do not develop, or do not swim, or do not fertilize or some combination of these spermatozoan catastrophes. Numerous contraceptive targets abound and several of these targets are worthy of further exploratory work, including blocking transmembrane ion currents (Kirichok et al., 2006; Brenton et al., 1996), disrupting Sertoli-germ cell adhesions (Cheng et al., 2002, 2005) and disruption of spermiogenesis by imino sugars (Walden et al., 2006).

Immunocontraception, which showed great promise for many years, has lost its appeal. Nevertheless, immunocontraception can be used as a strategy to discern the function of target molecules in the male. As an example, Eppin is an epididymal protease inhibitor that coats the surface of human spermatozoa. Eppin modulates PSA (prostate specific antigen, a serine protease) activity and the hydrolysis of semenogelin. Although Eppin modulates the hydrolysis of semenogelin by PSA, antibodies to Eppin do not inhibit PSA activity.

Ejaculate spermatozoa of monkeys and humans are coated with Eppin. On the surface of spermatozoa Eppin binds the protein semenogelin, which is secreted by the seminal vesicles during ejaculation. The Eppin-semenogelin complex is removed during liquefaction of semen during the first 30 minutes after ejaculation. Failure to remove semenogelin results in infertile spermatozoa. Studies of the interaction of Eppin and semenogelin, and their effect on human spermatozoa, are described, for example, in Wang, Z., Widgren, E. E., Sivashanmugam, P., O'Rand, M. G., and Richardson, R. T. 2005. Association of Eppin with semenogelin on human spermatozoa. Biology of Reproduction 72 (4): 1064-1070 (Dec. 8, 2004).

One strategy for developing new contraceptives is to immunize primates with specific sperm surface antigens and determine the effects of the immune response on the ejaculated spermatozoa of immunized males. Recent work on Eppin, (SPINLW1; serine protease inhibitor-like, with Kunitz and WAP domains-1) provides an example of the utility of the immunocontraceptive approach (O'Rand et al., 2004; Wang et al., 2005; O'Rand et al., 2006). A fertility study (O'Rand et al., 2004) demonstrated that effective and reversible male immunocontraception in primates is an obtainable goal. A high serum titer (>1:1000) sustained over several months achieved an effective level of contraception. Treatment of human spermatozoa with antibodies to Eppin derived from primates showed a decrease in motility of the treated spermatozoa (results shown in FIG. 1).

The data in FIG. 1 were obtained from the analysis of affinity purified anti-eppin antibodies. Compared to control, pre-immune IgG, there was a significant difference in the progressive motility of human sperm after treatment with anti-eppin antibodies as judged by a decrease in the total distance traveled by 70% (p<6.2×10⁻⁹) and the straight line distance by 95% (p<9.4×10⁻¹²), while the velocity decreased by 82% (p<5×10⁻¹⁵). At the same time the antibodies had the effect of increasing the bend angle between the straight-line vector (distance) and a turn, i.e. the back and forth movement of the head (tortuosity).

Consequently, in addition to whatever conclusions one may wish to draw about the feasibility of using immunocontraception, one can conclude from these studies that Eppin has an essential role in fertility.

Antibodies are prone to degradation in the stomach if orally administered, and for this reason, are commonly administered by injection. Because it is unlikely that male contraception will be viable if it requires routine injections, it would be advantageous to have small molecules that also inhibit Eppin-semenogelin binding. The present invention provides such compounds, an assay for identifying such compounds, and methods for their use.

### SUMMARY OF THE INVENTION

Compounds suitable for use in providing male contraception, an assay method for identifying such compounds, and methods of providing contraception using the compounds, are provided.

The compounds described herein inhibit Eppin-semenogelin binding, and inhibit forward motility of sperm in humans and other primates. The Eppin-semenogelin complex is on the surface of sperm. Useful compounds include those that a) bind to the binding site on Eppin for semenogelin (also referred to herein as Sg), or which bind to an allosteric position in a manner which inhibits semenogelin from binding, and which also mimic the effect of the semenogelin, namely to stop sperm from swimming. Ideally, those compounds which interfere with Eppin semenogelin binding bind with higher affinity to the active binding pocket than semenogelin. This will enable one to administer lower effective concentrations of the compounds than compounds that bind with lower binding affinity. Those compounds which bind in an allosteric manner are also, ideally, high affinity compounds, so that lower effective concentrations of these compounds can be administered as well.

The assays described herein identify compounds which both inhibit eppin-semenogelin binding and inhibit sperm motility. Previous studies on the antisera from the infertile monkeys revealed 2 linear B-cell epitopes of anti-Eppin, one in the N-terminal and one in the C-terminal. The C-terminal epitope was identified as TCSMFVYGGCQGNNNNFQ. Antibodies to this epitope inhibit sperm motility and semenogelin binding.

The Eppin-semenogelin in vitro binding assay can be conducted in a high throughput manner, for example, in 96-well plates. The assay measures recombinant semenogelin binding to recombinant Eppin, and allows testing of inhibition by peptides, antisera and various small molecules. Human seminal plasma which contains native Eppin will bind in the assay and displace recombinant Eppin.

The semenogelin binding site on Eppin and the C-terminal anti-Eppin epitope overlap. Accordingly, the assay has modeled the Eppin C-terminal for surface contours in the area of Sg binding, specifically looking for surface pockets. A pocket was found surrounded by Asn42, 43, 44, 45, which are critical residues in the anti-Eppin epitope (see epitope sequence above). A subset of the Maybridge database was used to look for small molecules that would fit into and/or around this pocket, based on a hypothesis that an appropriate small molecule would bind to Eppin, block its binding to semenogelin and substitute for the antibody, i.e. block sperm motility. A series of seven such small molecules were identified and their docking in the pocket was modeled.

Of these compounds, one of them (H₂N—C(O)—CH₂—NH⁺—CH₂C(O)—NH₂ which we call A4) has a molecular weight of 166 Daltons, and is a small organic compound that mimics the effect of anti-Eppin antibodies. Compound A4 inhibited Eppin-semenogelin binding in the in vitro assay. When tested on human spermatozoa, this molecule inhibits their motility, significantly reducing their forward velocity and distance traveled. This compound reduces the percent motile spermatozoa in an ejaculate to infertility levels.

Thus, the assay involves two main steps. The first is to identify compounds that inhibit Eppin-semenogelin binding, and the second is to test such compounds on human spermatozoa (or other primate spermatozoa) to determine whether the compounds inhibit forward motility of the spermatozoa. An optional step, performed between the first and second step, is to determine whether the compounds inhibit semenogelin from binding. That is, certain compounds bind Eppin in the same site as semenogelin (as opposed to an allosteric position), and bind more tightly than semenogelin, thus inhibiting formation of an Eppin-semenogelin complex. Other compounds bind Eppin in an allosteric position, and inhibit Eppin-semenogelin complex formation in that manner. Either embodiment will work to inhibit spermatozoa forward motility

Compounds that inhibit Eppin-semenogelin binding, inhibit semenogelin from binding to Eppin, and inhibit spermatozoa forward motility can be used to temporarily and reversibly cause male infertility. The compounds can be included in compositions, which ideally are oral or transdermal compositions, which release an appropriate amount of the compounds to produce this effect. For example, the compounds can be used in once-daily tablets or pills, or transdermal patches for periods of time longer than a day, much in the same manner as female contraceptives.

In another embodiment, the compounds can be used in addition to, or in place of, spermicides in spermicidal compositions, such as those used in conjunction with condoms, diaphragms, and spermicidal jellies. That is, since the compounds can function on contact with spermatozoa to inhibit forward motility, it is not necessary that they be ingested to have the effect.

Thus, the invention described herein provides an advantage over the prior art, in that the user has a choice of male contraception between in vivo activity of the compounds to inhibit the forward motility of spermatozoa before ejaculation, or use of the compounds after ejaculation to inhibit forward motility of the spermatozoa.

The present invention will be better understood with reference to the following detailed description.

### BRIEF DESCRIPTION OF THE FIGURES

FIG. 1 is a chart showing the effect on the distance traveled, and velocity, of human spermatozoa treated with anti-Eppin-antibodies from infertile monkeys.

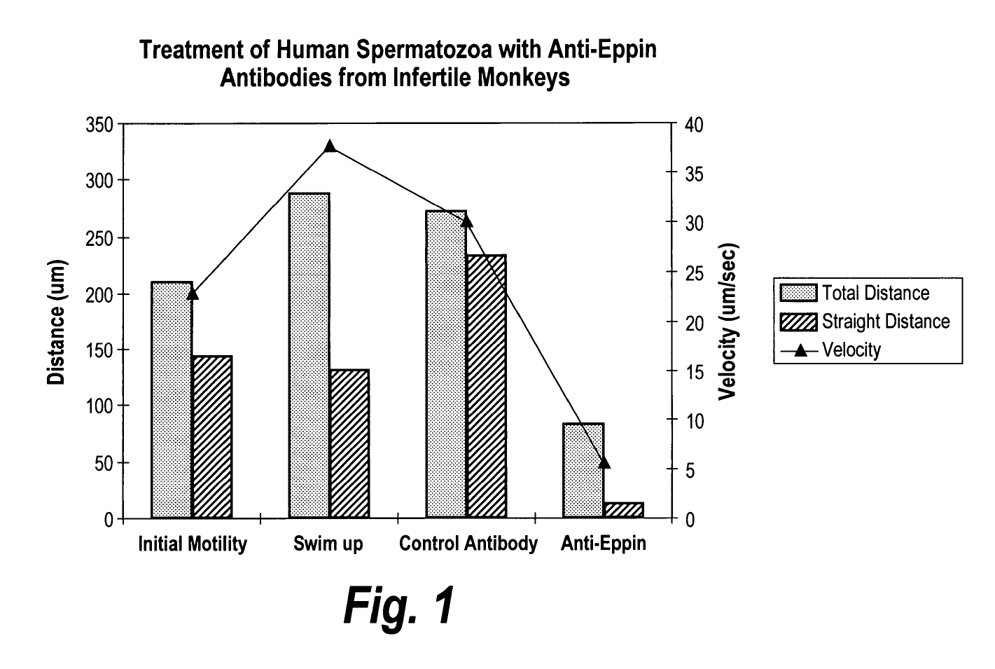

FIG. 2 is a graph showing the binding of compound A4 in the binding pocket of Eppin and semenogelin, as shown in an in silico model.

FIG. 3 is a chart showing one embodiment of a high throughput assay for identifying compounds that inhibit the binding of Eppin to semenogelin in an in vitro model.

FIG. 4 is a chart showing the linear correlation between the inhibition of Eppin-semenogelin binding (as shown by optical density (OD) at a wavelength of 450 nm) and concentration of compound A4 (μg/mL).

FIG. 5 is a chart showing the inhibition of spermatozoa motility by the compound A4, in terms of velocity (μm/sec) and distance traveled (μm) over time (hours).

FIG. 6 is a chart showing the inhibition of spermatozoa motility by the compound A4, in terms of velocity (μm/sec) and percent motility.

### DETAILED DESCRIPTION

Compounds suitable for use in providing male contraception, an assay method for identifying such compounds, and methods of providing contraception using the compounds, are provided.

The present invention will be better understood with reference to the following definitions.

### DEFINITIONS

Eppin (SPINLW1; serine protease inhibitor-like, with Kunitz and WAP domains 1) is a member of the whey acidic protein (WAP)-type four-disulfide core (WFDC) gene family. The WFDC genes are on human chromosome 20q12-q13 in two clusters, one centromeric and one telomeric (Clauss et al., 2002). Eppin is WFDC 7 in the telomeric cluster and is the archetype of WFDC genes characterized by encoding both Kunitz-type and WAP-type four disulfide core protease inhibitor consensus sequences (Richardson et al., 2001).

Eppin is a testis/epididymal specific protein (Richardson et al., Gene 270, 93 (2001) and Sivashanmugam, et al., Gene 312, 125 (2003). The human Eppin gene on chromosome 20 encodes two isoforms, one with and one without a secretory signal sequence, each containing both a Kunitz-type and a WAP-type (four-disulfide core) protease inhibitor consensus sequence. Eppin represents the first member of a family of protease inhibitors on human chromosome 20 characterized by dual inhibitor consensus sequences (ibid). There are three splice variants of Eppin that are expressed differently; Eppin-1 is expressed in the testis and epididymis, Eppin-2 is expressed in the epididymis and Eppin-3 in the testis.

The preparation of recombinant human Eppin (rhEppin) has been described in detail (Wang et al., Biology of Reproduction, 72:1064-1070 (2005) and the rhEppin used in the examples described herein lacks part of the N-terminal secretory signal sequence as described in Wang et., ibid. Briefly, rhEppin was prepared in E. coli strain M15 [pREP-4]and the protein purified from the bacterial lysate on a Ni²⁺-NTA column (Qiagen, Valencia, Calif.; 21). Purified rhEppin was extensively dialyzed against phosphate buffered saline (PBS, pH 7.2) before use. The recombinant Eppin used in the assays described herein has been tagged with a FLAG tag.

Semenogelin I (SgI) and semenogelin II (SgII) are the dominating protein components of the coagulum formed by freshly ejaculated human semen. These proteins are primarily found in the seminal vesicles, although SgII is produced in small amounts in the epididymis. These proteins have not been detected in other tissues Lundwall et al., Mol. Hum. Reprod. 8(9):805-10 (September 2002).

Recombinant semenogelin, as used herein, is a His-tagged recombinant protein, such as that describe in Wang et al., Biology of Reproduction, 72:1064-1070 (2005), the contents of which are hereby incorporated by reference, which first described Eppin-semenogelin binding. In this paper, two recombinant fragments of semenogelin are described, which were identified via polymerase chain reaction (PCR). The fragment starting with amino acid 164 and extending to amino acid 283 binds to Eppin. This fragment can be appropriately His-tagged and used in the assays described herein.

The terms “active ingredient” or “active agent” mean compounds which inhibit Eppin-semenogelin binding and inhibit spermatozoa forward motility, as well as any prodrugs thereof and pharmaceutically acceptable salts, hydrates, and solvates of the compound and the prodrugs. The term “other ingredients” means any excipients, diluents, binders, lubricants, carriers, surfactants, and mixtures thereof that are formulated with metanicotines or any prodrugs thereof and pharmaceutically acceptable salts, hydrates, and solvates thereof.

The term “appropriate period of time” or “suitable period of time” means the period of time necessary to achieve a desired effect or result. For example, a mixture can be blended until a potency distribution is reached that is within an acceptable range for a given application or use of the blended mixture.

“Carriers” or “vehicles” as used herein refer to carrier materials suitable for drug administration, specifically including oral and transdermal administration, and include any such materials known in the art, e.g., any liquid, gel, solvent, liquid diluent, solubilizer, or the like, which is non toxic and which does not interact with other components of the composition in a deleterious manner. Examples of suitable vehicles for use in transdermal formulations include water, alcohols such as isopropyl alcohol and isobutyl alcohol, polyalcohols such as glycerol, and glycols such as propylene glycol, and esters of such polyols, (e.g., mono-, di-, or tri-glycerides).

By “controlled” is meant reduced or minimized peak and valley exposure cycles in blood, plasma, or other biological fluids normally present in some routes of administration of a pharmacologically active agent.

An “effective” or an “adequate” permeation enhancer for transdermal formulations as used herein means a permeation enhancer that will provide the desired increase in skin permeability and correspondingly, the desired depth of penetration, rate of administration, and amount of drug delivered.

The term “effective amount,” as used herein means the amount determined by such considerations as are known in the art for causing temporary and reversible male contraception.

“Penetration enhancement” or “permeation enhancement” as used herein in connection with transdermal administration relates to an increase in the permeability of skin to a pharmacologically active agent, i.e., so as to increase the rate at which the drug permeates through the skin (i.e., flux) and enters the bloodstream. The enhanced permeation effected by using these enhancers can be observed by measuring the rate of diffusion (or flux) of drug through animal or human skin or a suitable polymeric membrane using a diffusion cell apparatus as described in the examples herein.

By “transdermal” delivery, applicants intend to include both transdermal (or “percutaneous”) and transmucosal administration, i.e., delivery by passage of a drug through the skin or mucosal tissue and into the bloodstream.

By “sustained” is meant extended maintenance of steady state plasma levels of an active compound.

The term “unit dose,” “unit dosage,” or “unit dosage form” means a physically discrete unit that contains a predetermined quantity of active ingredient calculated to produce a desired therapeutic effect. The dosage form can be in any suitable form for administration, such as oral or transdermal administration, which forms are well known to those of skill in the art.

In some embodiments, the active compounds are present in the form of amines, and their pharmaceutically acceptable salts. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, benzoate, and ascorbate; salts with amino acids such as lysine monohydrochloride, aspartate and glutamate. The salts may be in some cases hydrates or ethanol solvates. The salts can be prepared by reacting an active compound as described herein with a suitable acid. For transdermal administration, it can be preferred that the acid is a fatty acid, to form a salt that has relatively easy transmission through the skin.

In any embodiments described herein, the active blend of a dosage form generally includes one or more pharmaceutically acceptable adhesives, excipients, carriers, diluents, binders, lubricants, glidants, or disintegrants and depends upon the purpose for which the active ingredient is being applied. In general, transdermal formulations are made of other ingredients including, but not limited to, excipients, diluents, carriers, permeation enhancers, and mixtures thereof.

### I. High Throughput Assay

The ability of compounds to inhibit Eppin-semenogelin binding, as well as their ability to inhibit sperm motility, can be assayed in a high throughput manner.

Co-immunoprecipitation (co-IP) is a widely used in vitro method for protein-protein interaction (PPI) discovery. This affinity-based molecular pull-down method, based on the epitope tagging of recombinant Eppin and semenogelin proteins, allows one to rapidly evaluate the ability of small molecules to interfere with the binding of the recombinant Eppin and semenogelin proteins.

There are commercially available (i.e., from Sigma-Aldrich and others) 96-well plate-based immunoprecipitation platforms, based on the FLAG® epitope tag (DYKDDDDK), that enable one to conduct highly efficient high-throughput assays. The ANTI-FLAG® M2 antibody coated 96-well plate provides a rapid system for quantitative analysis of captured FLAG-tagged fusion protein complexes via an ELISA format.

Compared to traditional resin-based IP, the multi-well (i.e., 96-well) format allows one to evaluate large numbers of compounds for their ability to inhibit Eppin-semenogelin binding, as well as numerous variables such as time points, side-by-side analysis of controls, replicate samples, and the like.

In one embodiment, His-tagged recombinant semenogelin is bound to the individual wells of the plate, FLAG-tagged recombinant Eppin is bound to the semenogelin. The recombinant Eppin and recombinant semenogelin will form a complex. Compounds are then individually added to the wells of the plate and allowed to interfere with the Eppin-semenogelin complex formation. An anti-FLAG antibody (such as the M2 antibody sold by Sigma-Aldrich) is then added to the wells of the plate. The inhibition of the binding of Eppin to semenogelin is determined by looking for changes in Eppin-semenogelin binding when the anti-FLAG antibody (such as those used in commercially-available ELISA assays) is added. One can detect the binding of the anti-FLAG antibody to the FLAG tagged Eppin, for example, by looking at the absorption at 450 nm. In another embodiment, compounds are incubated with FLAG-tagged recombinant Eppin before being added to Sg. The anti-FLAG antibody is added after the Eppin-compound mixtures are added to the wells of the plate.

Putative compounds that potentially interfere with Eppin-semenogelin binding can be (ideally, individually) added to the individual wells, and allowed to remain in contact with the recombinant Eppin-semenogelin complex. Those compounds which inhibit binding will affect the absorption at 450 nm, making it easy to detect compounds that inhibit such binding.

The activity of compounds that are identified as being capable of interfering with the binding of Eppin and semenogelin (i.e., their ability to inhibit motility of human spermatozoa) can be determined in a multi-well fashion, or in individual in vitro assays with live human spermatozoa.

The motility of the spermatozoa can be measured using means known to those of skill in the art. Two methods commonly used in the past to gauge sperm motility are high speed photography and subsequent subjective film analysis and the use of a visual inspection of a semen sample using a graticule and stop watch technique. Laser spectroscopy, together with subsequent computer analysis, has also been used to yield estimates of live/dead sperm ratios, concentration measurements, the distribution of head rotation rates and the distribution of forward velocities. Another method for measuring sperm motility is described in U.S. Pat. No. 4,601,578, the contents of which are hereby incorporated by reference. In this method, a micro-sample of semen is exposed to a beam of light from a laser device. Light scattered by the sample is detected by a photodetector. The photodetector signal is analyzed in the frequency domain to provide a measure of the amplitude of the signal components having frequencies above about 100 Hz. This measure is representative of the numbers of motile sperm in the sample. A motile/immotile percentage can be obtained by dividing the motile measurement by a value representative of the amplitude of the full-frequency photodetector signal. Frequency-amplitude analysis can be carried out in the time domain, for example, by using frequency splitting filters and integrating the filtered signals.

In one embodiment, a computer program is used to manage the assay, by recording the identity of the compounds, and the activity of the compounds. In this manner, a series of high-throughput multi-well plates can be screened, and the identity of compounds that inhibit Eppin-semenogelin binding stored for later use. Such computer programs are well known to those of skill in the art of high throughput screening.

### II. Compounds Identified in the High-Throughput Assay

The compounds described herein inhibit Eppin-semenogelin binding, and inhibit forward motility of sperm in humans and other primates. The Eppin-semenogelin complex is on the surface of sperm. Useful compounds include those that a) bind to the binding site on Eppin for semenogelin (also referred to herein as Sg), or which bind to an allosteric position in a manner which inhibits semenogelin from binding, and which also mimic the effect of the semenogelin, namely to stop sperm from swimming. Ideally, those compounds which interfere with Eppin semenogelin binding bind with higher affinity to the active binding pocket than semenogelin. This will enable one to administer lower effective concentrations of the compounds than compounds that bind with lower binding affinity. Those compounds which bind in an allosteric manner are also, ideally, high affinity compounds, so that lower effective concentrations of these compounds can be administered as well.

Compounds identified in the high-throughput assay as inhibiting the binding of Eppin and semenogelin, and also identified as having a negative impact on spermatozoa motility, can be used in the methods described herein.

One compound identified in the high-throughput assay described above, which both inhibits the binding of Eppin and semenogelin, and which inhibits the motility of human spermatozoa, is referred to herein as Compound A4. The structure of this compound is provided below:

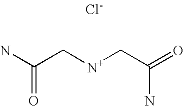

This compound is also active in the form of other pharmaceutically acceptable salts, as defined herein, or in its free base form.

Additional compounds that can inhibit Eppin-semenogelin binding, have the following formula: R₂NC(O)—CHR′—N⁺CHR′C(O)NR₂ X⁻, where R is selected from the group consisting of H, alkyl, aryl, arylalkyl, and alkylaryl, or two R groups on a nitrogen can form a C₅₋₆ cycloalkyl ring, R′ is an alkyl, aryl, arylalkyl, or alkylaryl group, halo (i.e., F, Cl, Br, or I), amine, thiol, hydroxyl, carboxyl, nitro, fluoroalkyl, such as trifluoromethyl,), and X⁻ is a pharmaceutically acceptable salt, as defined herein,

where alkyl groups are defined as C₁₋₆ alkyl groups,

aryl groups include C₅₋₁₀ aryl or heteroaryl rings, such as benzene, thiophene, pyrollidine, naphthalene, pyridine, pyrimidine, and,

alkyl-aryl is a C₁₋₆ alkyl group bound to a C₅₋₁₀ aryl or heteroaryl ring, such as a benzyl group, and

aryl-alkyl is a C₅₋₁₀ aryl or heteroaryl ring bound to a C₁₋₆ alkyl group.

In one embodiment, at least one R group on the carbon is a C₁₋₆ alkyl, such as methyl, which inhibits the ability of monoamine oxidase to degrade the compound, thus enabling the compound to have a relatively longer in-vivo half-life.

### III. Pharmaceutical Compositions Including the Compounds

Temporary and reversible male contraception can be achieved by administering to the patient an effective amount of the compounds described above, in the presence of a pharmaceutically acceptable carrier or diluent, using any of the modes of administration as described in detail herein. Of course, the treatment is not necessarily reversible on any one ejaculate, but once one stops taking/using the compound, the effect on sperm motility will cease and the infertility will be reversed.

The active materials can be administered by any appropriate route, for example, orally, parenterally, enterally, intravenously, intradermally, subcutaneously, transdermally, intranasally or topically, in liquid or solid form. Ideally, the compounds are delivered orally or transdermally, to ensure maximal patient compliance.

The active compounds are included in the pharmaceutically acceptable carrier or diluent in an amount sufficient to deliver to a patient a therapeutically effective amount of compound to inhibit sperm motility without causing serious toxic effects in the treated patient. By “inhibitory amount” is meant an amount of active ingredient sufficient to exert an inhibitory effect on sperm motility as measured by, for example, an assay such as the ones described herein.

A preferred dose of the compound for all the above-mentioned conditions will be in the range from about 1 to 75 mg/kg, preferably 1 to 20 mg/kg, of body weight per day, more generally 0.1 to about 100 mg per kilogram body weight of the recipient per day. The effective dosage range of the compounds can be calculated by means known to those skilled in the art.

The compounds are conveniently administered in unit any suitable dosage form, including but not limited to one containing 7 to 3000 mg, preferably 70 to 1400 mg of active ingredient per unit dosage form. An oral dosage of 50 to 1000 mg is usually convenient.

The concentration of active compound in the drug composition will depend on absorption, distribution, metabolism and excretion rates of the drug as well as other factors known to those of skill in the art. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible bind agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compounds can be administered as a component of an elixir, suspension, syrup, wafer, chewing gum or the like. A syrup may contain, in addition to the active compounds, sucrose as a sweetening agent and certain preservatives, dyes and colorings and flavors.

Solutions or suspensions used for parental, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

Controlled Release Formulations

All of the U.S. patents cited in this section on controlled release formulations are incorporated by reference in their entirety.

The field of biodegradable polymers has developed rapidly since the synthesis and biodegradability of polylactic acid was reported by Kulkarni et al., in 1966 (“Polylactic acid for surgical implants,” Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers). See also U.S. Pat. No. 5,626,863 to Hubbell, et al which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and PCT WO 97/05185 filed by Focal, Inc. directed to multiblock biodegradable hydrogels for use as controlled release agents for drug delivery and tissue treatment agents.

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et. al.; (1991) “Surface modification of polymeric biomaterials for reduced thrombogenicity,” Polym. Mater. Sci. Eng., 62:731 735]).

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a nonreactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

U.S. Pat. No. 5,641,745 to Elan Corporation, plc discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 to Elan Corporation plc describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. No. 5,505,962 describes a controlled release pharmaceutical formulation.

Transdermal Delivery

The compositions for transdermal administration include the active compounds, or their pharmaceutically acceptable salts, including fatty acid salts, and optionally can also include other ingredients including, but not limited to, carriers and excipients, such as permeation enhancers which promote transdermal absorption of the active ingredient after transdermal administration.

Relative to an oral dosage form such as a tablet or capsule, transdermal delivery can provide both more rapid or more sustained absorption, controlled release and therefore controlled onset of therapeutic action, and avoidance of liver first pass metabolism. For patients who have difficulty in swallowing tablets, capsules or other solids or those who have intestinal failure, the transdermal delivery route can be preferred. This route of administration can also be preferred for its ease of use, and for patients who otherwise might forget to take once-daily pills.

The amount of drug absorbed depends on many factors. These factors include the drug concentration, the drug delivery vehicle, skin contact time, the area of the skin dosed, the ratio of the ionized and unionized forms of the drug at the pH of the absorption site, the molecular size of the drug molecule, the drug's relative lipid solubility, and the relative affinity of the drug for the skin versus the formulation (if drug is not readily released from its formulation matrix, very little drug absorption will be realized). Those of skill in the art can readily prepare an appropriate transdermal composition, which delivers an appropriate amount of the active agent, taking these factors into consideration.

Transdermal Devices

The transdermal device for delivering the compounds described herein can be of any type known in the art, including the monolithic, matrix, membrane, and other types typically useful for administering drugs by the transdermal route. Such devices are disclosed in U.S. Pat. Nos. 3,996,934; 3,797,494; 3,742,951; 3,598,122; 3,598,123; 3,731,683; 3,734,097; 4,336,243; 4,379,454; 4,460,372; 4,486,193; 4,666,441; 4,615,699; 4,681,584; and 4,558,580, 5,533,995, among others; the disclosures of which are incorporated herein by reference.

These devices tend to be flexible, adhere well to the skin, and have a polymeric backing (covering) that is impermeable to the drug to be delivered, so that the drug is administered through the skin. The drug, or pharmaceutically acceptable salt thereof, is typically present in a solution or dispersion, which can be in the form of a gel, and which aids in drug delivery through the stratum corneum of the epidermis and to the dermis for absorption.

Membrane Devices

Membrane devices typically have four layers: (1) an impermeable backing, (2) a reservoir layer, (3) a membrane layer (which can be a dense polymer membrane or a microporous membrane), and (4) a contact adhesive layer which either covers the entire device surface in a continuous or discontinuous coating or surrounds the membrane layer. Examples of materials that may be used to act as an impermeable layer are high, medium, and low density polyethylene, polypropylene, polyvinylchloride, polyvinylidene chloride, polycarbonate, polyethylene terepthalate, and polymers laminated or coated with aluminum foil. Others are disclosed in the standard transdermal device patents mentioned herein. In certain embodiments in which the reservoir layer is fluid or is a polymer, the outer edge of the backing layer can overlay the edge of the reservoir layer and be sealed by adhesion or fusion to the diffusion membrane layer. In such instances, the reservoir layer need not have exposed surfaces.

The reservoir layer is underneath the impermeable backing and contains a carrier liquid, typically water and/or an alcohol, or polyol or ester thereof, and may or may not contain the active compounds. The amount of drug in the reservoir depends on the desired rate of absorption through the skin from the device and the intended duration of therapy. The reservoir layer can include diluents, stabilizers, vehicles, gelling agents, and the like in addition to the carrier liquid and active compounds.

The diffusion membrane layer of the laminate device can be made of a dense or microporous polymer film that has the requisite permeability to the drug and the carrier liquid. Preferably, the membrane is impermeable to ingredients other than the drug and the carrier liquid, although when buffering at the skin surface is desired, the membrane should be permeable to the buffer in the formulation as well. Examples of polymer film that may be used to make the membrane layer are disclosed in U.S. Pat. Nos. 3,797,454 and 4,031,894. The preferred materials are polyurethane, ethylene/vinyl alcohol copolymers or ethylene/vinyl acetate.

Monolithic Matrices

The second class of transdermal systems is represented by monolithic matrices. Examples of such monolithic devices are U.S. Pat. No. 4,291,014, U.S. Pat. No. 4,297,995, U.S. Pat. No. 4,390,520, and U.S. Pat. No. 4,340,043. Others are known to those of ordinary skill in this art.

Monolithic and matrix type barrier transdermal devices typically include:

(1) Porous polymers or open-cell foam polymers, such as polyvinyl chloride (PVC), polyurethanes, polypropylenes, etc.

(2) Highly swollen or plasticized polymers such as cellulose, HEMA or MEMA or their copolymers, hydroxypropyl methylcellulose (HPMC), hydroxyethyl methylcellulose (HEMC), and the like, polyvinyl alcohol (PVA)/polyvinylpyrollidone (PVP), or other hydrogels, or PVC, polyurethane, ethylene/vinyl acetate, or their copolymers.

(3) Gels of liquids, typically including water and/or hydroxyl-containing solvents such as ethanol, and often containing gelling agents such PVP, carboxymethylcellulose (CMC), Klucel, HPMC, alginates, kaolinate, bentonite, or montmorillonite, other clay fillers, stearates, silicon dioxide particles, etc.

(4) Nonwoven materials made of textiles, celluloses, polyurethanes, polyester or other fiber.

(5) Sponges, which can be formed from natural or foamed polymers.

(6) Adhesives, ideally dermatologically-acceptable pressure sensitive adhesives, for example, silicone adhesives or acrylic adhesives.

The various components for the transdermal formulations are described in more detail below.

Polymeric Barrier Materials

Representative polymeric barrier materials include, but are not limited to:

Polycarbonates, such as those formed by phosgenation of a dihydroxy aromatic such as bisphenol A, including materials are sold under the trade designation Lexan® (the General Electric Company);

Polyvinylchlorides, such as Geon® 121 (B. G. Goodrich Chemical Company);

Polyamides (“nylons”), such as polyhexamethylene adipamide, including NOMEX® (E. I. DuPont de Nemours & Co.).

Modacrylic copolymers, such as DYNEL®, are formed of polyvinylchloride (60 percent) and acrylonitrile (40 percent), styrene-acrylic acid copolymers, and the like.

Polysulfones, for example, those containing diphenylene sulfone groups, for example, P-1700 (Union Carbide Corporation).

Halogenated polymers, for example, polyvinylidene fluoride, such as Kynar® (Pennsalt Chemical Corporation), polyvinylfluoride, such as Tedlar® (E. I. DuPont de Nemours & Co.), and polyfluorohalocarbons, such as Aclar® (Allied Chemical Corporation).

Polychlorethers, for example, Penton® (Hercules Incorporated), and other thermoplastic polyethers.

Acetal polymers, for example, polyformaldehydes, such as Delrin® (E. I. DuPont de Nemours & Co.).

Acrylic resins, for example, polyacrylonitrile, polymethyl methacrylate (PMMA), poly n-butyl methacrylate, and the like.

Other polymers such as polyurethanes, polyimides, polybenzimidazoles, polyvinyl acetate, aromatic and aliphatic, polyethers, cellulose esters, e.g., cellulose triacetate; cellulose; colledion (cellulose nitrate with 11% nitrogen); epoxy resins; olefins, e.g., polyethylene, polypropylene; polyvinylidene chloride; porous rubber; cross linked poly(ethylene oxide); cross-linked polyvinylpyrrolidone; cross-linked poly(vinyl alcohol); polyelectrolyte structures formed of two ionically associated polymers of the type as set forth in U.S. Pat. Nos. 3,549,016 and 3,546,141; derivatives of polystyrene such as poly(sodium styrenesulfonate) and poly(vinylbenzyltrimethyl-ammonium chloride); poly(hydroxyethylmethacrylate); poly(isobutylvinyl ether), and the like, may also be used. A large number of copolymers which can be formed by reacting various proportions of monomers from the above list of polymers are also useful.

If the membrane or other barrier does not have a sufficiently high flux, the thickness of the membrane or barrier can be reduced. However, the thickness should not be reduced to the point where it is likely to tear, or to a point where the amount of drug which can be administered is too low.

Adhesives

The transdermal drug delivery formulations typically include a contact adhesive layer to adhere the device to the skin. The active agent may, in some embodiments, reside in the adhesive.

Exemplary adhesives include polyurethanes; acrylic or methacrylic resins such as polymers of esters of acrylic or methacrylic acid with alcohols such as n-butanol, n-pentanol, isopentanol, 2-methylbutanol, 1-methylbutanol, 1-methylpentanol, 2-methylpentanol, 3-methylpentanol, 2-ethylbutanol, isooctanol, n-decanol, or n-dodecanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tertbutylacrylamide, itaconic acid, vinylacetate, N-branched alkyl maleamic acids wherein the alkyl group has 10 to 24 carbon atoms, glycol diacrylates, or mixtures of these; natural or synthetic rubbers such as styrenebutadiene, butylether, neoprene, polyisobutylene, polybutadiene, and polyisoprene; polyvinylacetate; unreaformaldehyde resins; phenolformaldehyde resins; resorcinol formaldehyde resins, cellulose derivatives such as ethylcellulose, methylcellulose, nitrocellulose, cellulose acetatebutyrate, and carboxymethyl cellulose; and natural gums such as guar, acacia, pectins, starch, dextrin, albumin, gelatin, casein, etc. The adhesives can be compounded with tackifiers and stabilizers, as is well known in the art.

Representative silicone adhesives include silicone elastomers based on monomers of silanes, halosilanes, or C₁₋₁₈ alkoxysilanes, especially polydimethylsiloxanes which may be used alone or formulated with a silicone tackifier or silicone plasticizer which are selected from medically acceptable silicone fluids, i.e. non-elastomeric silicones based on silanes, halosilanes or C₁₋₁₈ alkoxysilanes. Typical silicone adhesives are available from Dow Corning under the tradename SILASTIC®.

Liquid Vehicles

Transdermal formulations can include a variety of components, including a liquid vehicle, typically a C₂₋₄ alkanol such as ethanol, isopropanol, n-propanol, butanol, a polyalcohol or glycol such as propylene glycol, butylene glycol, hexylene glycol, ethylene glycol, and/or purified water. The vehicle is typically present in an amount of between about 5 and about 75% w/w, more typically, between about 15.0% and about 65.0% w/w, and, preferably, between about 20.0 and 55.0% w/w. Water augments the solubility of hydrophilic active agents in the formulation, and accelerates the release of lipophilic active agents from a formulation. Alcohols, such as ethanol, increase the stratum corneum liquid fluidity or function to extract lipids from the stratum corneum. As discussed herein, the glycols can also act as permeation enhancers.

Permeation Enhancers

Successful transdermal delivery depends on sufficient flux of the drug across skin, and sufficient surface area of skin, to produce an efficacious plasma concentration of the drug. For reasons of consumer acceptance, the practical surface area of a transdermal system is limited from approximately 5 to 100 cm². With this limitation on surface area, the therapeutic transdermal administration of many drugs requires an increase in the inherent skin permeability to obtain the necessary flux. Accordingly, compounds have been developed which enhance percutaneous absorption of the drugs to be administered.

Permeation enhancers are described, for example, in U.S. Pat. Nos. 5,785,991; 4,764,381; 4,956,171; 4,863,970; 5,453,279; 4,883,660; 5,719,197, and in the literature “Pharmaceutical Skin Penetration Enhancement”, J. Hadgraft, Marcel Dekker, Inc. 1993; “Percutaneous Absorption”, R. Bronaugh, H. Maibach, Marcel Dekker, Inc. (1989), B. W. Barry, “Penetration Enhancers in Skin Permeation”, Proceedings of the 13th international Symposium on Controlled Release of Bioactive Materials, ed. by Chaudry & Thies, Controlled Release Society, Lincolnshire, Ill., pp. 136-137 (1986), and Cooper & Berner, “Penetration Enhancers”, in The Transdermal Delivery of Drugs, Vol. II ed. by Kydonieus and Berner, CRC Press, Boca Raton, Fla. pp. 57-62 (1986), the contents of each of which are hereby incorporated by reference.

The permeation enhancers should both enhance the permeability of the skin, and be non-toxic, non-irritant and non-sensitizing on repeated exposure. Representative permeation enhancers include, for example, sucrose monococoate, glycerol monooleate, sucrose monolaurate, glycerol monolaureate, diethylene glycol monoalkyl ethers such as diethylene glycol monoethyl or monomethyl ether (Transcutol® P), ester components such as propylene glycol monolaurate, methyl laurate, and lauryl acetate, monoglycerides such as glycerol monolaurate, fatty alcohols such as lauryl alcohol, and 2-ethyl-1,3 hexanediol alone or in combination with oleic acid.

In one embodiment, the transdermal compositions are provided with skin permeation enhancing benefits by combining the active compounds with saturated fatty alcohols, or forming salts of the compounds with one or more fatty acids, such as those of the formula CH₃—(CH₂)ₙ—CH₂OH or CH₃—(CH₂)ₙ—CH₂COOH respectively, in which n is an integer from 8 to 22, preferably 8 to 12, most preferably 10 or an unsaturated fatty alcohol or fatty acid given by the formula CH₃—(CₙH₂₍ₙ₋ₓ₎)—OH or CH₃—(CₙH₂₍ₙ₋ₓ₎)—COOH respectively in which n is an integer from 8 to 22 and x is the number of double bonds; and preferably also a second component that is a monoalkyl ether of diethylene glycol, preferably diethylene glycol monoethyl ether or diethylene glycol monomethyl ether, in a vehicle or carrier composition, integrated by an C₁₋₄ alkanol, preferably ethanol; a polyalcohol, preferably propylene glycol and purified water.

A binary system including a combination of oleic acid or oleic alcohol and a lower alcohol, or a combination of a lower alkyl ester of a polycarboxylic acid, an aliphatic monohydroxy alcohol and an aliphatic diol, can be used.

Representative permeation enhancers include fatty alcohols and fatty acids, and monoalkyl ethers of diethylene glycol such as diethylene glycol monoethyl ether or diethylene glycol monomethyl ether. The fatty alcohols are typically present in an amount of between about 0.1 and about 20.0% w/w, preferably between about 0.2 and about 10.0% w/w, and more preferably, between about 0.4 and about 3.0% w/w. The diethylene glycol monoalkyl ethers are typically present in an amount up to 40.0% w/w, preferably between about 0.2 and 25.0% w/w, and, more preferably, between about 2.0 and about 8.0% w/w.

Although not wishing to be bound to a particular theory, it is believed that the mechanism by which certain permeation enhancers function to enhance permeability of the active agents through the stratum corneum is as follows:

The fatty alcohol is mainly distributed to the stratum corneum because of its lipophilicity and interacts with the stratum corneum lipids.

The diethylene glycol monoalkyl ethers dissolve both hydrophilic and a lipophilic active agents therein, and facilitates the penetration of the active agents to the skin.

Glycols, such as propylene glycol, act as a cosolvent of the active agents, and thus increase their solubility in the formulation. Further, they tend to solvate the intracellular keratin of the stratum corneum, and thus enhance drug mobility and skin hydration.

Gelling Agents

Gelling agents, such as carbomer, carboxyethylene or polyacrylic acid such as Carbopol® 980 or 940 NF, 981 or 941 NF, 1382 or 1342 NF, 5984 or 934 NF, ETD 2020, 2050, 934P NF, 971P NF, 974P NF, Noveon® AA-1 USP, etc; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel®, different grades), hydroxyethylcellulose (HEC) (Natrosol® grades), HPMCP 55, Methocel® grades, etc; natural gums such as arabic, xanthan, guar gums, alginates, etc; polyvinylpyrrolidone derivatives such as Kollidon® grades; polyoxyethylene polyoxypropylene copolymers such as Lutrol® F grades 68, 127, etc; others like chitosan, polyvinyl alcohols, pectins, veegun grades, et, can also be present. Those of the skill in the art know of other gelling agents or viscosants suitable for use in the present invention.

Representative gelling agents include, but are not limited to, Carbopol® 980 NF, Lutrol® F 127, Lutrol® F 68 and Noveon® AA-1 USP. The gelling agent is present from about 0.2 to about 30.0% w/w, depending on the type of polymer.

Preservatives

The transdermal compositions can also include one or more preservatives and/or antioxidants. Representative preservatives include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenylethyl alcohol; organic acids or salts thereof such as benzoic acid, sodium benzoate, potassium sorbate, parabens; or complex forming agents such as EDTA. Representative antioxidants include butylhydroxytoluene, butylhydroxyanisole, ethylenediaminetetraacetic acid and its sodium salts, D,L-alpha tocoferol.

Other Components

Other components may include diluents such as cellulose, microcrystalline cellulose, hydroxypropyl cellulose, starch, hydroxypropylmethyl cellulose and the like. Excipients can be added to adjust the tonicity of the composition, such as sodium chloride, glucose, dextrose, mannitol, sorbitol, lactose and the like. Acidic or basic buffers can also be added to control the pH. Co-solvents or solubilizers such as glycerol, polyethylene glycols, polyethylene glycols derivatives, polyethylene glycol 660 hydroxystearate (Solutol HS15 from BASF), butylene glycol, hexylene glycol, and the like, can also be added.

Controlled Release of the Active Agent

The administration of the active agent can be controlled by using controlled release formulations, which can provide rapid or sustained release, or both, depending on the formulations.

There are numerous particulate drug delivery vehicles known to those of skill in the art which can include the active ingredients, and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active agent is in particulate form without added components, its release rate depends on the release of the active agent itself. In contrast, if the active agent is in particulate form as a blend of the active agent and a polymer, the release of the active agent is controlled, at least in part, by the removal of the polymer, typically by dissolution or biodegradation.

In some embodiments, the compositions can provide an initial rapid release of the active ingredient followed by a sustained release of the active ingredient. U.S. Pat. No. 5,629,011 provides examples of this type of formulation and is incorporated herein by reference in its entirety. There are numerous transdermal compositions that use transdermal delivery to deliver nicotine in a time-release manner (such as rate-controlling membranes), including currently marketed therapies for female contraception and nicotine replacement. These are also suitable for administering the compounds described herein.

### IV. Methods of Providing Reversible and Temporary Male Contraception

The compounds and compositions for oral or transdermal administration can be used to provide reversible and temporary male contraception. Because the compounds function by inhibiting the binding of Eppin and semenogelin, once administration of the compounds is terminated, the inhibition of this binding will be terminated, thus restoring fertility.

In one aspect, the compounds are administered orally, preferably in a once-daily format, to provide male contraception. In another aspect, the compounds are administered transdermally, ideally in a lesser frequency, such as once weekly or once monthly, to provide male contraception.

Those of skill in the art can effectively follow the administration of these therapies without undue experimentation. Until an appropriate dosage is determined for an individual patient, it may be desirable to have the patient take the dosage for a particular amount of time, for example, a week or a month, with periodic measurement of sperm motility, to ensure that the dosage for the particular patient is the right dosage. As with female contraception, the compositions may be provided in several different dosage levels, to provide an optimal dosage for each patient. The optimal dosage is, ideally, one which reliably provides contraception, but does not significantly exceed the dosage required to achieve such contraception.

### V. Spermicidal Compositions

For certain individuals, it can be equally important to prevent transmission of disease as it is to prevent pregnancy. Thus, it can be desirable to use the compounds described herein to inhibit spermatozoa forward motility (thus inhibiting fertilization) in conjunction with a condom.

For certain other individuals, there is a desire to prevent pregnancy, a desire (in the individual or in their partner) to avoid taking an oral contraceptive agents, and a desire to avoid using condoms. For these individuals, it can be desirable to use the compounds described herein to inhibit spermatozoa forward motility (thus inhibiting fertilization) in conjunction with a diaphragm, female condom, or spermicidal lubricant or jelly.

In these embodiments, the compositions may include other agents, such as those that locally inhibit viral, fungal, and/or bacterial infections.

Lubricant Compositions

In one embodiment, the compounds described herein are present in prophylactic lubricant compositions for use during sexual relations. The lubricant compositions can be used without condoms, female condoms, or diaphragms, but when used in conjunction with such devices, can provide an additional safety margin, in case the conventional devices are defective.

The lubricant compositions typically include an effective lubricant, an effective concentration of the active compound, and, optionally, an antimicrobial compound effective in destroying the human immunodeficiency virus (HIV, such as HIV-1 and HIV-2), herpes virus, human pappiloma virus (HPV), hepatitis B or C(HBV and HCV) and other viruses and/or bacteria, such as syphilis, gonorrhea, or Chlamydia, or fungi, such as those which cause yeast infections. The antimicrobial compound can also be a spermicide which acts in a manner differently than the instant compounds (i.e., does not inhibit Eppin-semenogelin binding or inhibit spermatozoa forward motility). For example, certain compounds react with the vaginal mucosa to form a barrier to the penetration of sperm cells into the uterus, without substantial detrimental side effects.

The prophylactic lubricant compositions can also include a fungicide, such as methylparaben.

One representative antimicrobial compound is chlorhexidine and its salts, particularly the gluconate or digluconate salts. Chlorhexidine diffuses into the cervical mucous, creating a suspension that restricts penetration of sperm cells during ovulation and also causes them to rapidly lose their mobility. This occurs at concentrations of chlorhexidine in excess of 0.1%. Thus, by using chlorhexidine as the active antimicrobial compound in accordance with the present invention, the chlorhexidine diffuses into the cervical mucous prior to the ejection of semen and in effect creates the “sealed bag” of the vagina, which will retain all the body secretions including the semen. Any viruses present can also be destroyed by the chlorhexidine, which, in concentrations above 0.1%, effectively destroys the envelope of the virus and in so doing prevents the virus from penetrating the human cell.

Thus, lubricant that includes the compounds described herein and an antimicrobial compound can be applied to the sex organs to reduce the friction between the penis and the vaginal wall, kill any bacteria and viruses in the body fluids, and inhibit sperm motility (and possibly also destroy sperm).

Ideally, the pH of the lubricant compositions approximates that of the vaginal tissues, since these tissues could otherwise be inhibited from regenerating if the pH of the lubricant is significantly outside of this normal range.

The lubricant may be any effective lubricant or combination of lubricants acceptable for cosmetic applications. The lubricant compositions can include an alcohol or mixtures of alcohols, and are preferably water-soluble so they can be used with condoms, female condoms and diaphragms, and also to minimize any stains that might form on clothing or sheets. Any water-soluble lubricants can be used, though preferred lubricants include glycerol and propylene glycol. Representative water-soluble lubricants include those in Ky-jelly and other water-based lubricant compositions.

The present invention will be better understood with reference to the following non-limiting examples.

### Example 1

### In Silico Assay to Identify Compounds which Inhibit Eppin-Semenogelin Binding

An in silico assay was developed based on the putative binding site of Eppin and semenogelin. This site is at the C-terminal end of Eppin, namely amino acids 75-133. This amino acid sequence was incorporated into a model using the Sybyl modeling program, which enables one to view the three dimensional structure of the binding site and screen in silico libraries of compounds for their ability to bind the putative binding site. Hits identified using this in silico assay can then be tested in an in vitro assay, such as the ones in Examples 2 and 3 that follow.

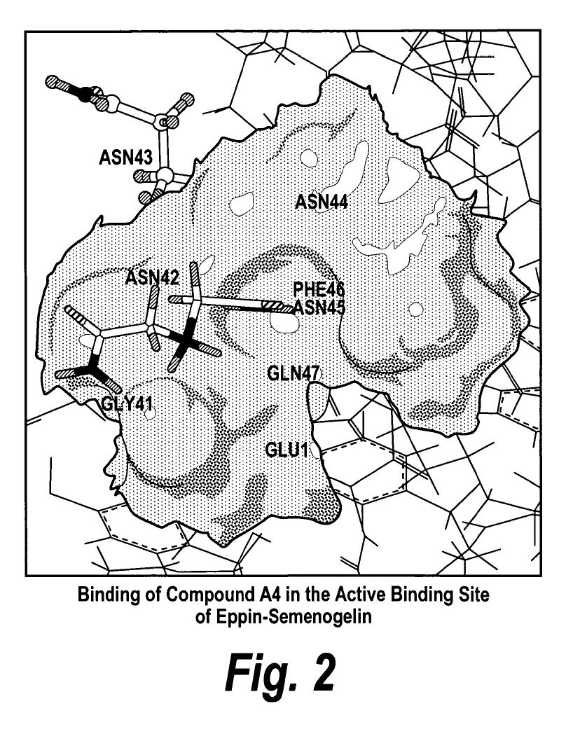

When a library of small molecules was evaluated for their ability to bind to Eppin and block its binding to Sg, seven small molecules were identified and modeled for their docking in the pocket. A representative docking of a small molecule in the binding pocket in Eppin is shown in FIG. 2.

FIG. 2 is a molecular model of the C-terminal of Eppin demonstrating the pocket around the infertile monkey anti-eppin antibody binding site (O'Rand et al., 2004) and the semenogelin binding site. FIG. 2 also shows compound A4 docked in the semenogelin-eppin binding site modeled on the C-terminal of eppin.

Of the 7 small molecules modeled on Eppin, 6 (A1-A6) were ordered and tested in vitro using the in vitro assay in Example 2.

### Example 2

### High Throughput Assay to Identify Compounds which Inhibit Eppin-Semenogelin Binding

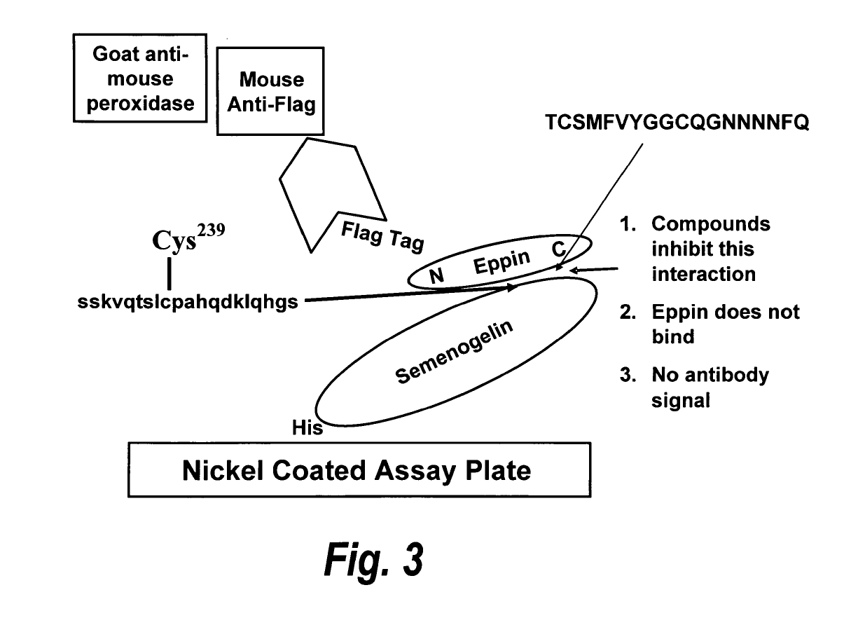

The compounds identified in Example 1 were tested in an in vitro assay for their ability to inhibit the binding of Eppin and semenogelin. The assay used is shown schematically in FIG. 3.

Background

The goal of this assay was to evaluate whether compounds would bind to Eppin, and inhibit the binding of Eppin to semenogelin. In an alternative embodiment, the Eppin could be bound to semenogelin, and the ability of the compounds to dislodge semenogelin can be measured.

The former assay is perhaps more useful in identifying compounds for administration to a patient interested in temporary and reversible infertility, because the compound would be administered, cross the epididymis, come in contact with Eppin, and thus bind Eppin. The thus-bound Eppin would not come into contact with semenogelin until ejaculation, and since it would be bound to the administered compound, it would not bind to semenogelin to form the Eppin semenogelin complex. Thus, the sperm would remain immotile.

The latter assay is perhaps more useful in identifying compounds for use in spermicidal lubricants, because the compound would come into contact with the Eppin after ejaculation (i.e., after the Eppin had already formed a complex with semenogelin). The ability to break up the Eppin/semenogelin complex would be the relevant factor in this case, so this assay would identify compounds that had this function, thus rendering sperm immotile after ejaculation and after Eppin/semenogelin complex formation.

Materials

Recombinant Eppin and semenogelin are as described in Wang et al., Biology of Reproduction, 72:1064-1070 (2005). The recombinant Eppin used in the assays described herein has been tagged with a FLAG tag. The recombinant semenogelin is a His-tagged recombinant protein of the fragment of semenogelin starting with amino acid 164 and continuing to amino acid 283.

A subset of the Maybridge database was tested in Example 1, and compounds A1 and A4 were evaluated in this assay.

Methods

The first step was to prepare preincubated samples of Eppin with compounds at various dilutions, starting at 40 μg/ml. Eppin (1 μg) was incubated with dilutions of A1 and A4 in 1× Casein for 2 hours at r.t. The preincubated samples (150 μl of preincubated samples/well) were transferred into 6 His plates previously coated with 100 ng/well HisSg.

The Eppin, semenogelin, and compounds were incubated overnight at 4 degrees, and the plates were washed (3×PBST (phosphate-buffered saline [PBS] buffer containing 0.05% Tween 20) and soaked for five minutes. Anti-M2 antibody (150 μl/well, 1:1000 in 1× Casein) was added to each well, and allowed to incubate for one hour. The wells were then washed (3× in PBST, 5 minute soak). A solution of GAM-HRP (goat anti-mouse horseradish peroxidase) in 1× Casein/PBST (1:1000) was prepared, and pipetted into each well at a concentration of 150 μl/well. The mixture was incubated for 1 hr at r.t. The wells were then washed (3×PBST, 5 min soak, 2×PBS), and TMB substrate was added (200 μl/well). The mixtures were then incubated for 60 minutes, and the reaction was then stopped by adding 1N HCl (100 μl/well).

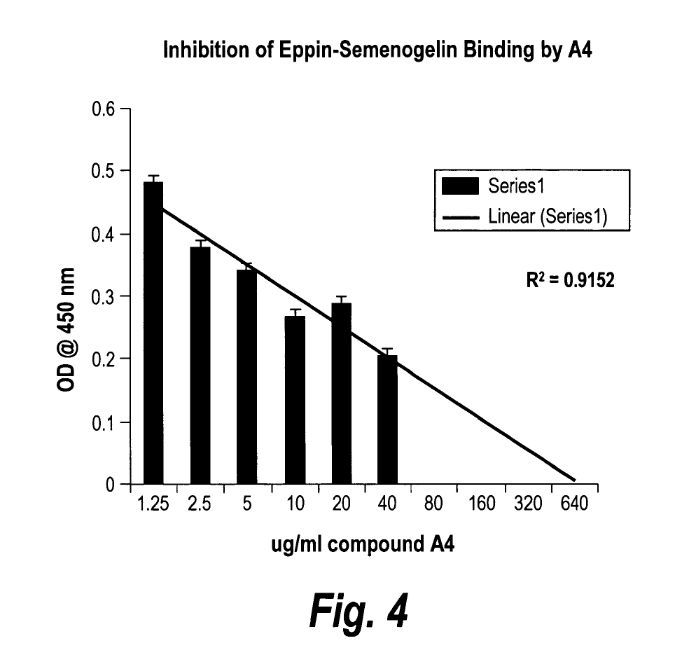

The compounds (at effective concentrations) that were able to inhibit Eppin semenogelin binding were identified by looking for absorption at 450 nm (due to the GAM-HRP). The results are shown in FIG. 4, which shows the effect of increasing amounts of A4 on Eppin-semenogelin binding. The data demonstrate that A4 inhibits Eppin binding to semenogelin.

It was determined that Compound A4 was effective at inhibiting this binding in a dose dependent manner, and Compound A4 was then evaluated in a second in vitro assay (Example 4) for its ability to inhibit sperm motility.

### Example 3

### Assay for Eppin-Sg Binding in a High Throughput Screen (HTS)

In another embodiment of a high-throughput screen to identify compounds effective at inhibiting the binding of Eppin to semenogelin, putative compounds can be screened using a variation on the AlphaScreen™ assay from Packard BioScience for HTS.

The AlphaScreen™ (AS) is amplified luminescent proximity homogeneous assay in which two small beads (200 nm) are employed to hold donor and acceptor protein molecules. The donor and acceptor molecules can be Eppin and semenogelin, and when these proteins bind, singlet state oxygen molecules diffuse from the donor bead to the acceptor bead (˜4 μsec) and fluorophores subsequently emit light at 520-620 nm.

This technology has been designed for high throughput screening, and can be adapted for Eppin-semenogelin binding and used in 384 well plates to screen compound databases.

Strongly positive hits can be retested in a dilution series, and those compounds which still remain positive can be examined for their aqueous solubility, lipophilicity (ability to go through the cell membrane) and chemical stability. Compounds with the potential for drug development (i.e. solubility, selectivity and potency) will be considered candidates for further drug development, and possible for lead optimization chemistry.

### Example 4

### Ability of Compound A4 to Inhibit Sperm Motility

Appropriate small molecules bind to Eppin and block its binding to Sg. A series of seven such small molecules were identified using the in silico assay of Example 1, and tested for their in vitro ability to inhibit Eppin/semenogelin binding in Example 2. Compound A4 (LT00112470, molecular weight=166 Daltons) inhibited Eppin-semenogelin binding in a dose dependent manner (FIG. 4).

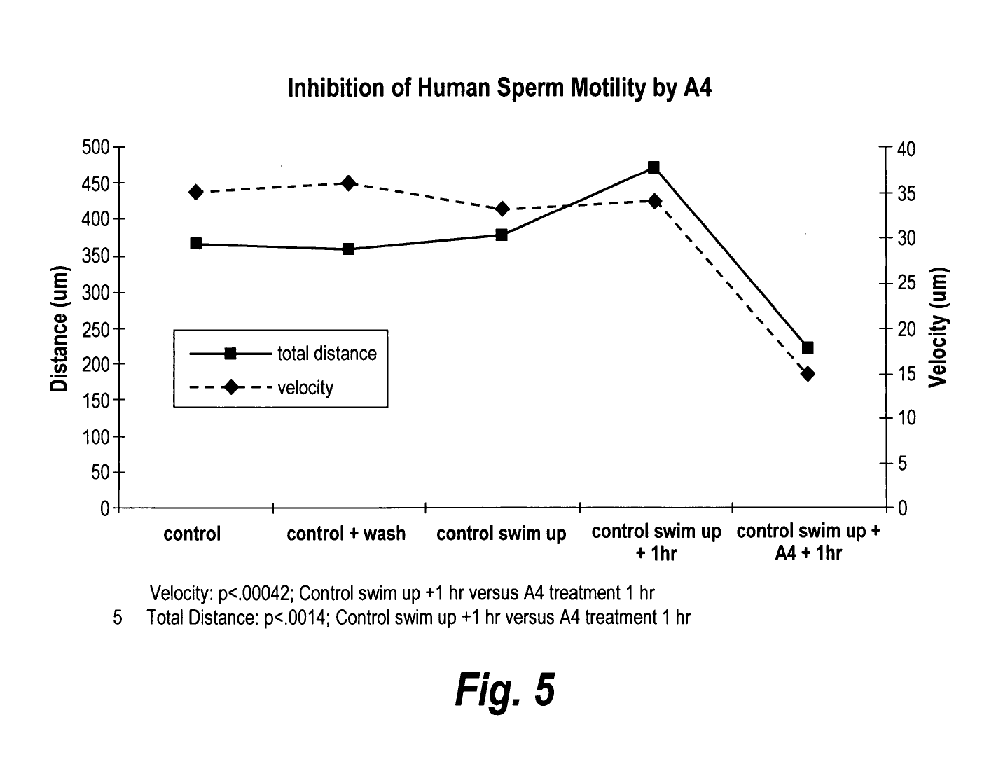

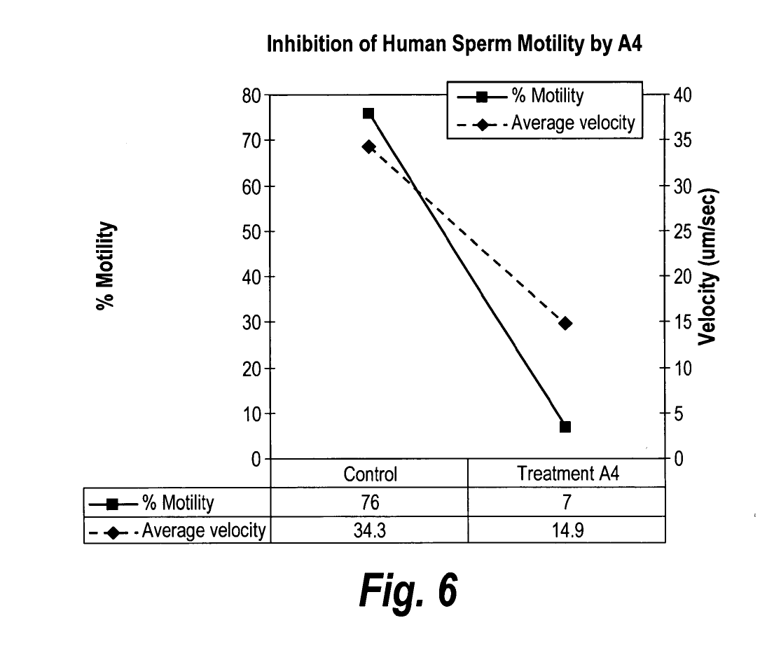

Human spermatozoa were obtained from the UNC-Hospital clinic, washed and used for testing in a motility assay. Compound A4 reduced the total number of motile sperm in an ejaculate to <10% within one hour. The data are shown below in FIGS. 5 and 6, and in Tables 2 and 3.

TABLE 2

| | total distance (μm) | Velocity (μm/sec) |
|---|---|---|
| control | 366 | 35 |
| control + wash | 359 | 36 |
| swim up | 378 | 33 |
| swim up + 1 hr | 471 | 34 |
| swim up + A4 + 1 hr | 222 | 15 |
| | p < .0014 | p < .0042 |

TABLE 3
Human sperm motility trial with Compound A4

| | | | Average | Straight | |
|---|---|---|---|---|---|
| | | Distance | velocity | distance | |
| | | (μm) | (μm/sec) | (μm) | % Motility |
| Control | 1 hour | 381.4 | 40.3 | 64.2 | |
| Control | swim up + | 470.8 | 34.3 | 68.2 | 76 |
| | 1 hour | | | | |
| Treatment A4 | swim up + | 222 | 14.9 | 48.7 | 7 |
| | 1 hour | | | | |

The data show that Compound A4 was effective at inhibiting sperm motility, and thus can be an effective male contraceptive.

While the foregoing specification teaches the principles of the present invention, with examples provided for the purpose of illustration, it will be understood that the practice of the invention encompasses all of the usual variations, adaptations and/or modifications as come within the scope of the following claims and their equivalents. All references cited herein are incorporated by reference in their entirety for all purposes.

1. A method of providing contraception, comprising administering an effective amount of a small molecule that inhibits Eppin/semenogelin binding, to a patient in need of male contraception.

2. The method of claim 1, wherein the small molecule has the formula:
R₂NC(O)—CHR′—N⁺CHR′C(O)NR₂ X⁻; where R is selected from the group consisting of H, alkyl, aryl, arylalkyl, and alkylaryl, or two R groups on a nitrogen can form a C₅₋₆ cycloalkyl ring, R′ is an alkyl, aryl, arylalkyl, or alkylaryl group, halo (i.e., F, Cl, Br, or I), amine, thiol, hydroxyl, carboxyl, nitro, fluoroalkyl, such as trifluoromethyl, and X⁻ is a pharmaceutically acceptable anion,
where alkyl groups are defined as C₁₋₆ alkyl groups,
aryl groups include C₅₋₁₀ aryl or heteroaryl rings, and
alkyl-aryl is a C₁₋₆ alkyl group bound to a C₅₋₁₀ aryl or heteroaryl ring, and
aryl-alkyl is a C₅₋₁₀ aryl or heteroaryl ring bound to a C₁₋₆ alkyl group.

3. The method of claim 2, wherein the small molecule has the formula:
(H₂N—C(O)—CH₂—NH⁺—CH₂C(O)—NH₂ X⁻; where X⁻ refers to a pharmaceutically acceptable anion.

4. A pharmaceutical composition for providing male contraception, comprising an effective amount of a small molecule that inhibits Eppin/semenogelin binding, and a pharmaceutically acceptable carrier or diluent.

5. The composition of claim 4, wherein the small molecule has the formula:
R₂NC(O)—CHR′—N⁺CHR′C(O)NR₂ X⁻; where R is selected from the group consisting of H, alkyl, aryl, arylalkyl, and alkylaryl, or two R groups on a nitrogen can form a C₅₋₆ cycloalkyl ring, R′ is an alkyl, aryl, arylalkyl, or alkylaryl group, halo (i.e., F, Cl, Br, or I), amine, thiol, hydroxyl, carboxyl, nitro, fluoroalkyl, such as trifluoromethyl, and X⁻ is a pharmaceutically acceptable salt,
where alkyl groups are defined as C₁₋₆ alkyl groups,
aryl groups include C₅₋₁₀ aryl or heteroaryl rings, and
alkyl-aryl is a C₁₋₆ alkyl group bound to a C₅₋₁₀ aryl or heteroaryl ring, and
aryl-alkyl is a C₅₋₁₀ aryl or heteroaryl ring bound to a C₁₋₆ alkyl group.

6. The composition of claim 4, wherein the small molecule has the formula:
(H₂N—C(O)—CH₂—NH⁺—CH₂C(O)—NH₂ X⁻; where X⁻ refers to a pharmaceutically acceptable anion.

7. A spermicidal composition, comprising a water-soluble lubricant and an effective amount of a small molecule that inhibits Eppin/semenogelin binding.

8. The composition of claim 7, wherein the small molecule has the formula:
R₂NC(O)—CHR′—N⁺CHR′C(O)NR₂ X⁻, where R is selected from the group consisting of H, alkyl, aryl, arylalkyl, and alkylaryl, or two R groups on a nitrogen can form a C₅₋₆ cycloalkyl ring, R′ is an alkyl, aryl, arylalkyl, or alkylaryl group, halo (i.e., F, Cl, Br, or I), amine, thiol, hydroxyl, carboxyl, nitro, fluoroalkyl, such as trifluoromethyl, and X⁻ is a pharmaceutically acceptable anion,
where alkyl groups are defined as C₁₋₆ alkyl groups,
aryl groups include C₅₋₁₀ aryl or heteroaryl rings, and
alkyl-aryl is a C₁₋₆ alkyl group bound to a C₅₋₁₀ aryl or heteroaryl ring, and
aryl-alkyl is a C₅₋₁₀ aryl or heteroaryl ring bound to a C₁₋₆ alkyl group.

9. The composition of claim 8, wherein the small molecule has the formula: (H₂N—C(O)—CH₂—NH⁺—CH₂C(O)—NH₂ X⁻; where X⁻ refers to a pharmaceutically acceptable anion.
